(19) United States
(12) Patent Application Publication
(10) Pub. No.: US 2019/0059863 A1
(43) Pub. Date: Feb. 28, 2019

(54) EXCHANGEABLE NEEDLE SAFETY MECHANISM

(71) Applicant: Boston Scientific Scimed, Inc., Maple Grove, MN (US)

(72) Inventors: Austin G. Johnson, Hudson, MA (US); Seamus F. O'Shaughnessy, Chelmsford, MA (US); Jeremy DiTullio, Worcester, MA (US); Routha Sim, Lowell, MA (US)

(21) Appl. No.: 16/115,935

(22) Filed: Aug. 29, 2018

Related U.S. Application Data
(60) Provisional application No. 62/552,230, filed on Aug. 30, 2017.

(51) Int. Cl. A61B 10/02

(52) U.S. Cl. CPC A61B 10/0233; A61B 17/3496

(57) ABSTRACT

The present disclosure relates generally to the field of medical devices. In particular, the present disclosure relates to medical devices and methods that include needle guards configured to shield the sharpened distal end of exchangeable biopsy needles. The biopsy needles may be used for tissue biopsy and sampling under radial ultrasound guidance.

### CROSS REFERENCE TO RELATED APPLICATIONS

This application claims the benefit of priority under 35 U.S.C. § 119 to U.S. Provisional Patent Application Ser. No. 62/552,230, filed on Aug. 30, 2017, which is incorporated by reference in its entirety for all purposes.

### FIELD

The present disclosure relates generally to the field of medical devices. In particular, the present disclosure relates to medical devices and methods that include needle guards configured to shield the sharpened distal end of exchangeable biopsy needles. The biopsy needles may be used for tissue biopsy and sampling under radial ultrasound guidance

### BACKGROUND

As an example of systems utilizing exchangeable needles, endoscopic ultrasound biopsy systems, such as the Expect™ and Expect Slimline systems (Boston Scientific Corp., Marlborough Mass.) and Beacon™ system (Medtronic, Minneapolis Minn.), allow biopsy samples to be obtained from potentially cancerous pulmonary nodules under radial ultrasound guidance. These systems include exchangeable needles to eliminate the need for the entire system to be removed from the patient, e.g., through the endoscope working channel, after each biopsy sample is taken. Exchangeable needles improve procedure time by allowing a second biopsy sample to be taken while the previous sample is expelled for analysis, and improve efficiency by maintaining the ultrasound image of the target nodule throughout the procedure. Once the needle is removed from the biopsy system, the exposed needle tip poses a puncture/needle-stick risk to medical professionals in the operating room and/or technicians in the diagnostic laboratory.

A variety of advantageous medical outcomes may therefore be realized by the devices and/or methods of the present disclosure, which provide the combined benefits of an exchangeable biopsy needle with needle guards that shield the exposed needle tip while still allowing the biopsy sample to be expelled for analysis.

### SUMMARY

In one aspect, the present disclosure relates to a needle guard comprising a housing. First and second flexible arms may extend distally from a proximal end of the housing. A gasket may be disposed within an opening in the housing and a hollow tube may be attached to the housing between the first and second flexible arms. A distal end of the first flexible arm may include a first tab, and a distal end of the second flexible arm may include a second tab. The gasket may include an opening configured to slidably receive an outer surface of a needle. The opening of the gasket may be configured to frictionally engage the other surface of the needle. At least a portion of the hollow tube may comprise an optically translucent material. The needle guard may further include a needle attachment component comprising a cylinder and a grip, wherein an inner surface of the cylinder may be configured to frictionally engage an outer surface of the housing. The first and second flexible arms may be configured to move between a first position and a second position. The first and second flexible arms may be configured to extend through an opening formed in a proximal end of a medical device. The first and second tabs may contact an inner surface of the medical device when the first and second arms are in the first position. The first and second tabs may not contact an inner surface of the medical device when the first and second arms are in the second position.

In another aspect, the present disclosure relates to a needle guard comprising a housing. A first wing may extend through a first opening in the housing, wherein a middle portion of the first wing may be pivotally attached to the housing and the first wing may be configured to move between a first position and a second position. A second wing may extend through a second opening in the housing, wherein a middle portion of the second wing may be pivotally attached to the housing and the second wing may be configured to move between a first position and a second position. A hollow tube may be attached to the housing. The housing and hollow tube may be configured to receive a needle therethrough. A surface of the first and second wings may not contact an outer surface of the needle when in the first position. A surface of the first and second wings may not contact the outer surface of the needle when in the second position. The first and second wings may lock a portion of the needle within the housing when in the second position. The portion of the needle locked within the housing may include a sharpened distal end of the needle.

In another aspect, the present disclosure relates to a needle guard comprising a housing. A single wing may extend through an opening in the housing, wherein a middle portion of the first wing may be pivotally attached to the housing and the single wing may be configured to move between a first position and a second position. A hollow tube may be attached to the housing. The housing and hollow tube may be configured to receive a needle therethrough. A surface of the single wing may not contact an outer surface of the needle when in the first position. The single wing may lock a portion of the needle within the housing when in the second position. The portion of the needle locked within the housing may include a sharpened distal end of the needle.

In yet another aspect, the present disclosure relates to a needle guard comprising a housing. A rod including a threaded surface may extend through an opening in the housing. An inner surface of the opening may include a grooved surface corresponding to the threaded surface. A hollow tube may be attached to the distal end of the housing. The housing and hollow tube may be configured to receive a needle therethrough. Rotating the rod in a first direction may move an end of the rod toward an interior of the housing, and rotating the rod in a second direction may move the end of the rod away from the interior of the housing. Rotating the rod in the first direction may place the end of the rod in contact with an outer surface of the needle.

### BRIEF DESCRIPTION OF THE DRAWINGS

Non-limiting embodiments of the present disclosure are described by way of example with reference to the accompanying figures, which are schematic and not intended to be drawn to scale. In the figures, each identical or nearly identical component illustrated is typically represented by a single numeral. For purposes of clarity, not every component is labeled in every figure, nor is every component of each embodiment shown where illustration is not necessary to allow those of ordinary skill in the art to understand the disclosure. In the figures:

FIGS. 1A-1B provide perspective views of medical device (FIG. 1A) and needle guard and needle attachment component (FIG. 1B), according to one embodiment of the present disclosure.

FIGS. 2A-2C provide perspective views of a needle guard, according to one embodiment of the present disclosure.

FIGS. 3A-3B provide perspective views of a needle guard, according to one embodiment of the present disclosure.

FIGS. 4A-4B provide perspective views of a needle guard, according to one embodiment of the present disclosure.

FIGS. 5A-5B provide perspective views of a needle guard, according to one embodiment of the present disclosure.

### DETAILED DESCRIPTION

The present disclosure is not limited to the particular embodiments described herein. The terminology used herein is for the purpose of describing particular embodiments only, and is not intended to be limiting beyond the scope of the appended claims. Unless otherwise defined, all technical terms used herein have the same meaning as commonly understood by one of ordinary skill in the art to which the disclosure belongs.

Although embodiments of the present disclosure are described with specific reference to medical devices and methods for acquiring biopsy samples from a pulmonary nodule under radial ultrasound guidance, it should be appreciated that such device and methods may be used in a variety of medical procedures where there is a need for exchangeable or single-use needles with needle guards that shield the exposed needle tip while still allowing the needle tip to be accessible, including, for example, in endoscopy procedures, intravenous procedures, etc.

As used herein, the singular forms “a,” “an,” and “the” are intended to include the plural forms as well, unless the context clearly indicates otherwise. It will be further understood that the terms “comprises” and/or “comprising,” or “includes” and/or “including” when used herein, specify the presence of stated features, regions, steps elements and/or components, but do not preclude the presence or addition of one or more other features, regions, integers, steps, operations, elements, components and/or groups thereof.

As used herein, the term “distal” refers to the end farthest away from the medical professional when introducing a device into a patient, while the term “proximal” refers to the end closest to the medical professional when introducing a device into a patient.

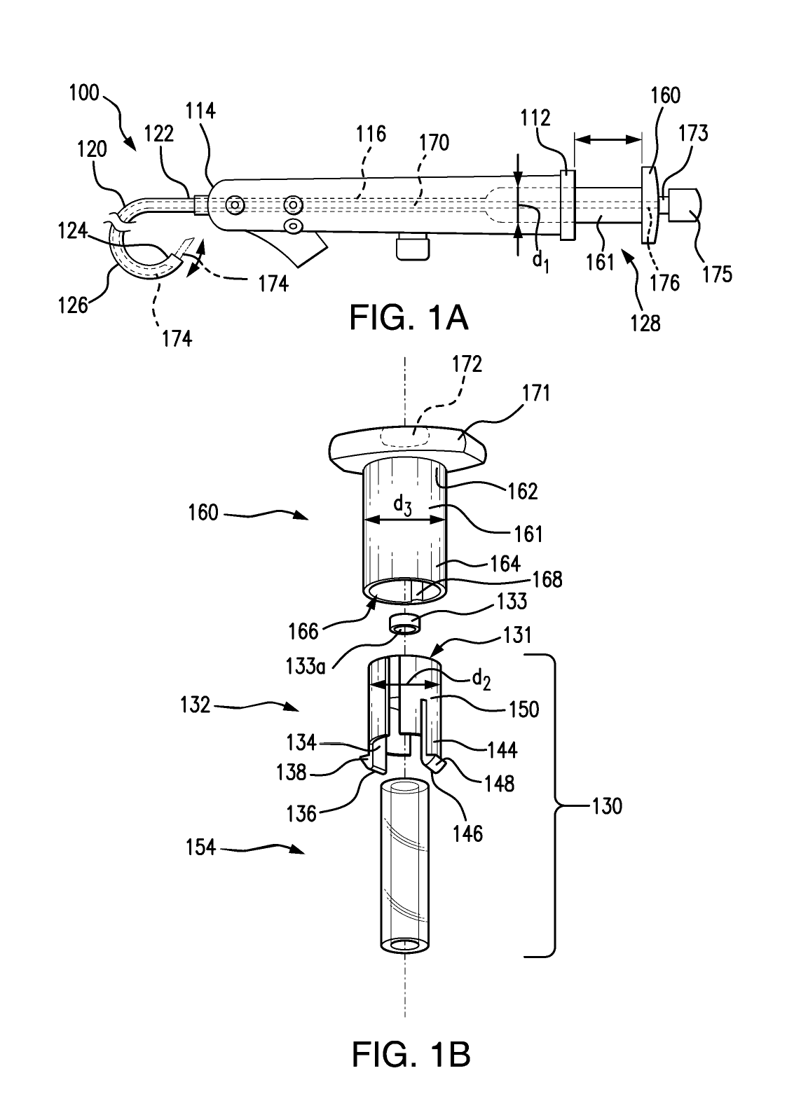

In various embodiments, the present disclosure relates to devices and methods to shield the exposed tip of an exchangeable biopsy needle following removal from the patient. Referring to FIG. 1A, in one embodiment, a medical device 100 of the present disclosure may include a handle 110 comprising a channel 116 extending between a proximal opening 112 and a distal opening 114. A catheter 120 comprising a proximal end 122, a distal end 124 and lumen 126 extending therebetween, may be attached to the handle 110 such that the lumen 126 is contiguous with the channel 116. The proximal opening 112 of the handle 110 may include an inner dimension d₁ sized and configured to receive at least a portion of a biopsy needle actuation mechanism 128.

Referring to FIG. 1B, in one embodiment, the biopsy needle actuation mechanism 128 may include a needle guard 130 and a needle attachment component 160. The needle guard 130 may include a housing 132 comprising an opening 131 extending therethrough. A gasket 133, or other friction creating component, may be disposed within the opening 131. The gasket 133 may include an opening 133a sized and configured to frictionally engage an outer surface of a biopsy needle, as discussed below. First and second flexible arms 134, 144 may extend distally from the housing 132. A distal end 136 of the first flexible arm 134 may include a tab 138, and a distal end 146 of the second flexible arm 144 may include a tab 148. The first and second flexible arms 134, 144 may be configured to move between a relaxed first position and a second position. For example, a force applied by the fingers of a user may compress the first and second flexible arms 134, 144 to deflect inward. The tabs 138, 148 may be configured to reversibly engage an inner surface of the handle of the medical device adjacent to the proximal opening 112, when the first and second flexible arms are in the first position.

The needle attachment component 160 may include a cylinder 161 comprising a proximal end 162, a distal end 164 and a lumen 166 extending therebetween. The cylinder 161 may include an outer dimension d₃ sized and configured to fit within the proximal opening 112 of the handle 110. A grip 171 may be attached to, or integrally formed with, the proximal end 162 of the cylinder 161. The grip 171 may extend substantially perpendicular to the cylinder 161 and include an opening 172 contiguous with the lumen 166. The housing 132 may include an outer dimension d₂ sized and configured to be frictionally received within the lumen 166 of the cylinder 161. For example, an outer surface 150 of the housing 132 may form an interference fit with an inner surface of the cylinder 161. In various embodiments, the interference fit may be sufficiently strong to prevent the needle guard 130 and needle attachment component 160 from separating from each other during a biopsy procedure (e.g., as the needle actuation mechanism is advanced and retracted within the proximal opening 112 of the handle 110), but sufficiently weak to allow separation, e.g., using the force applied by a user's fingers on the first and second flexible arms 134 and 144. In one embodiment, the outer surface 150 of the housing 132 may include a longitudinal groove or recess (not shown) configured to receive a corresponding longitudinal ridge or protrusion 168 on an inner surface of the cylinder 161. In addition to providing a friction or interference fit between the cylinder 161 and housing 132, the groove and protrusion 168 may provide a “keyed” fit to prevent rotation between the needle guard 130 and needle attachment component 160, and to maintain proper orientation of the biopsy needle, discussed below.

The needle guard 130 may further include an open-ended hollow tube 154 attached to, or integrally formed with, the housing 132. The hollow tube may extend distally beyond the distal ends 136, 146 of the first and second flexible arms 134, 144. At least a portion of the hollow tube 154 may comprise an optically translucent material (e.g., glass, plastic, rubber, etc.) to allow a user to visualize the sharpened end of a biopsy needle disposed therein, as discussed below.

Referring again to FIG. 1A, in one embodiment, a biopsy needle 170 comprising a proximal end 176 and a sharpened distal end 174 may be disposed within the medical device 100. For example, the sharpened distal end 174 may be inserted into the opening 172 formed within the grip 171 and advanced distally to pass through the lumen 166 of needle attachment component 160, the housing 132, gasket 133 and hollow tube 154 of the needle guard, the channel 116 of the handle 110 and the lumen 126 of the catheter 120. The proximal end 176 of the biopsy needle 170 may be attached to a needle hub 173 configured to secure or lock the biopsy needle 170 to the grip 171. The needle hub 173 may further include a stylet attachment 175 configured to removably secure a stylet (not shown) within the biopsy needle 170.

In one embodiment, the medical device 100 may be assembled by positioning the needle guard 130 within the proximal opening 112 of the handle 110 to place the tabs 138, 148 of the first and second flexible arms 134, 144 in contact with the inner surface of the handle 110 adjacent to the proximal opening 112. The needle attachment component 160 may then be secured to a portion of the housing 132 of the needle guard 130 extending proximally beyond the handle 110 by aligning the corresponding groove (not shown) and ridge 168, and advancing the cylinder 161 over the housing 132. A biopsy needle 170 and stylet (not shown) may then be advanced through the medical device 100 to position the sharpened distal end 174 within the catheter 120 at or near the distal end 124, and the needle hub 173 at the proximal end of the biopsy needle 170 secured to the grip 171.

In use, and by way of example, the catheter may be inserted into a pulmonary passage of a patient through a working channel of an endoscope and adjacent to a target pulmonary nodule. The stylet may then be removed and the biopsy needle 170 advanced from within the lumen 126 of the catheter 120 into the target pulmonary nodule by advancing/depressing the needle actuation mechanism 128 into the handle 110. For example, a user may grasp the handle 110 with their hand and depress the grip 171 with one or more fingers to move the cylinder 161 into and through the proximal opening 112. In one embodiment, the needle actuation mechanism 128 may be spring loaded and return to the non-deployed position when released by the user. The tabs 138, 148 on the first and second flexible arms 134, 144 may engage the inner surface of the handle 110 to prevent the needle actuation mechanism 128 and handle 110 from detaching when in the non-deployed position. The user may depress and release the needle actuation mechanism 128 as many times as necessary to acquire sufficient tissue for analysis.

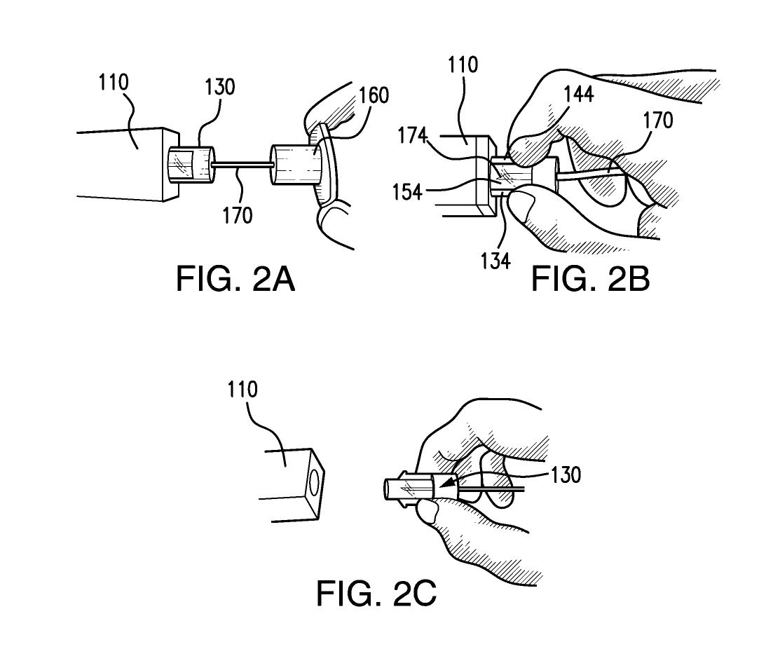

Referring to FIGS. 2A-2C, in one embodiment, when the user determines that the biopsy needle contains a sufficient amount of tissue from the target pulmonary nodule, the grip 171 may be proximally withdrawn with sufficient force to separate the needle attachment component 160 from the needle guard 130 (FIG. 2A). The needle attachment component 160 may be further proximally withdrawn until the sharpened distal end 174 of the biopsy needle is disposed within a portion of the hollow tube 154 of the needle guard extending proximally beyond the handle 110. The first and second flexible arms 134, 144 may then be depressed or squeezed, e.g. between a user's thumb and forefinger, to disengage the tabs 138, 148 (FIG. 1B) from the inner surface of the handle 110 (FIG. 2B) and remove the needle guard 130 from the handle 110 (FIG. 2C). In various embodiments, the gasket 133 (FIG. 1B) disposed within the housing 132 of the needle guard 130 may be configured to engage the outer surface of the biopsy needle 170 with sufficient frictional force to maintain the sharpened distal end 174 within the hollow tube 154. The tissue sample may then be expelled from the biopsy needle 170 while the sharpened distal end 174 remains safely housed within the hollow tube 154. In addition, or alternatively, the biopsy needle 170 may be advanced distally, e.g., along the hollow tube of the needle guard 130, if it is necessary to position the sharpened distal end 174 closer to, or distally beyond, the hollow tube 154.

Although the needle guard 130 of FIG. 1A includes first and second flexible arms 134, 144 extending from opposite sides of the housing 132, in various embodiments, the needle guard may include any number of arms disposed in a variety of patterns and/or configurations along or about the housing 132. In various other embodiments, the needle guard 130 may be removably attached to the handle 110 by corresponding threaded or luer-lock connectors, or the like.

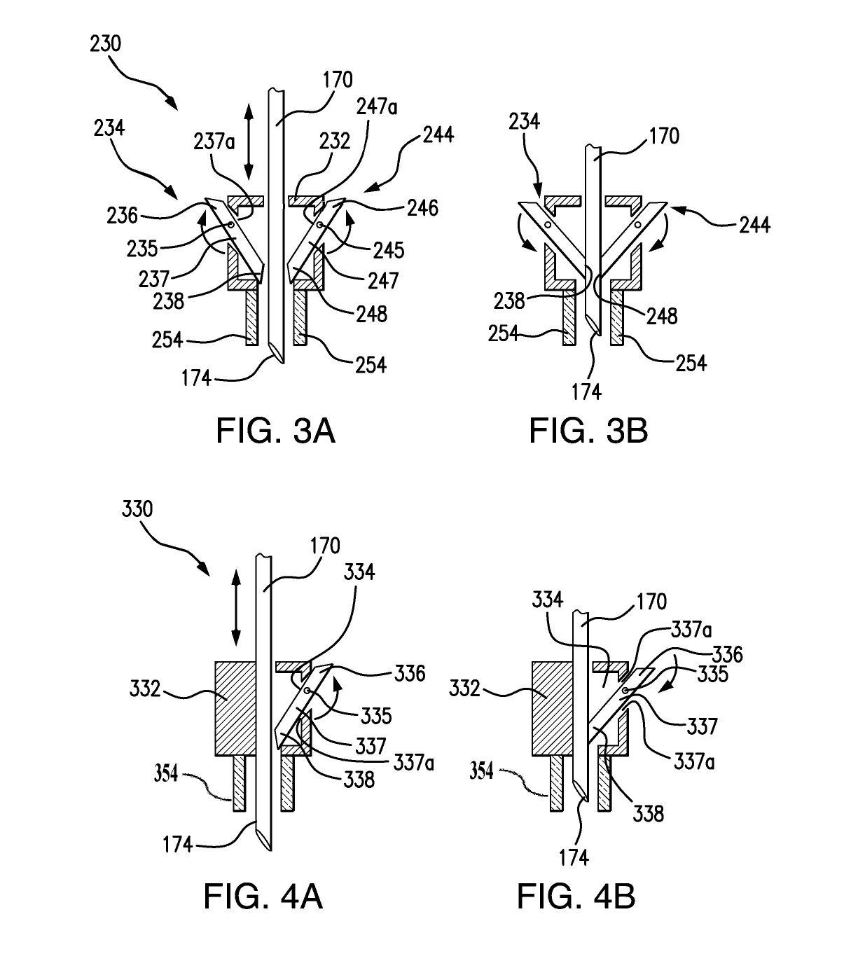

Referring to FIGS. 3A-3B, in one embodiment, a needle guard 230 of the present disclosure may include a housing 232 comprising first and second wings 234, 244 configured to maintain the sharpened distal end 174 of the biopsy needle 170 within an open-ended hollow tube 254 attached to or integrally formed with the housing 232. At least a portion of the hollow tube 254 may comprise an optically translucent material (e.g., glass, plastic, rubber, etc.) to allow a user to visualize the sharpened distal end of a biopsy needle disposed therein. The first wing 234 may include a first portion 236 disposed outside the housing 232, a second portion 238 disposed within the housing 232 and a middle portion 237 extending through an opening 237a formed within the housing 232. A first pin 235 may extend through the middle portion 237 to pivotally attach the first wing 234 to the housing 232. The second wing 244 may include a first portion 246 disposed outside the housing 232, a second portion 248 disposed within the housing 232 and a middle portion 247 extending through an opening 247a formed within the housing 232. A second pin 245 may extend through the middle portion 247 to pivotally attach the second wing 244 to the housing 232.

The first and second wings 234, 244 may be configured to pivot about the respective first and second pins 235, 245 between a first position (FIG. 3A) and a second position (FIG. 3B). For example, a user may actuate the first and second wings 234, 244 between the first and second positions by simultaneously depressing the first portions 236, 246 extending outside the housing 232. In the first position when the wings are not depressed, the biopsy needle 170 does not contact the first and second wings 234, 244, and is free to move/slide through the needle guard 230, e.g., as the needle attachment component and needle are withdrawn proximally to position the sharpened distal end 174 of the needle within the hollow tube 254. In the second position when the wings are depressed, a surface of the first and second wings 234, 244 may be placed in locked contact with an outer surface, e.g., opposite portions, of the biopsy needle 170. A force exerted between the opposed first and second wings 234, 244 may be sufficient to maintain the sharpened distal end 174 of biopsy needle 170 within the hollow tube 254 for safe removal of the tissue sample, as discussed above.

In various embodiments, at least a portion of the surface of the respective first and second wings 234, 244 may include an adhesive material (e.g., glue, adhesive tape), textured pattern (e.g., stippling, grooves, checkering, etc.), magnetic element, etc., which together with the pivot points (e.g., first and second pins 235, 245), may be configured to lock the biopsy needle 170 in place when the first and second wings 234, 244 are engaged and the biopsy needle is proximally retracted. Similarly, in one embodiment, the biopsy needle 170 and first and second wings 234, 234 may be disengaged (e.g., unlocked) by distally advancing the biopsy needle 170 without necessarily releasing the first and/or second wings 234, 244 by the user's fingers.

Referring to FIGS. 4A-4B, in one embodiment, a needle guard 330 of the present disclosure may include a housing 332 comprising a single wing 334 configured to maintain the sharpened distal end 174 of the biopsy needle 170 within an open-ended hollow tube 354 attached to or integrally formed with the housing 332. At least a portion of the hollow tube 354 may comprise an optically translucent material (e.g., glass, plastic, rubber, etc.) to allow a user to visualize the sharpened distal end of a biopsy needle disposed therein. The single wing 334 may include a first portion 336 disposed outside the housing 332, a second portion 338 disposed within the housing 332 and a middle portion 337 extending through an opening 337a formed within the housing 332. A pin 335 may extend through the middle portion 337 to pivotally attach the single wing 334 to the housing 332.

The single wing 334 may be configured to pivot about the pin 335 between a first position (FIG. 3A) and a second position (FIG. 3B). For example, a user may actuate the single wing 334 between the first and second positions by depressing the first portion 336 extending outside the housing 332. In the first position when the wing is not depressed, the biopsy needle 170 does not contact the single wing 334, and is free to move/slide through the needle guard 330, e.g., as the needle attachment component with the needle are withdrawn proximally to position the sharpened distal end 174 of the needle within the hollow tube 354. In the second position when the wing is depressed, a surface of the single wing 334 may be placed in locked contact with an outer surface of biopsy needle 170. A force exerted against the outer surface of the biopsy needle by the single wing 334 may be sufficient to maintain the sharpened distal end 174 of biopsy needle 170 within the hollow tube 354 for safe removal of the tissue sample, as discussed above.

In various embodiments, at least a portion of the surface of the single wing 334 may include an adhesive material (e.g., glue, adhesive tape), textured pattern (e.g., stippling, grooves, checkering, etc.), magnetic element, etc., which together with the pivot point (e.g., pin 335), may be configured to lock the biopsy needle 170 in place when the single wing 334 is engaged and the biopsy needle is proximally retracted. Similarly, in one embodiment, the biopsy needle 170 and single wing 334 may be disengaged (e.g., unlocked) by distally advancing the biopsy needle 170 without necessarily releasing the single wing 334 by the user's fingers.

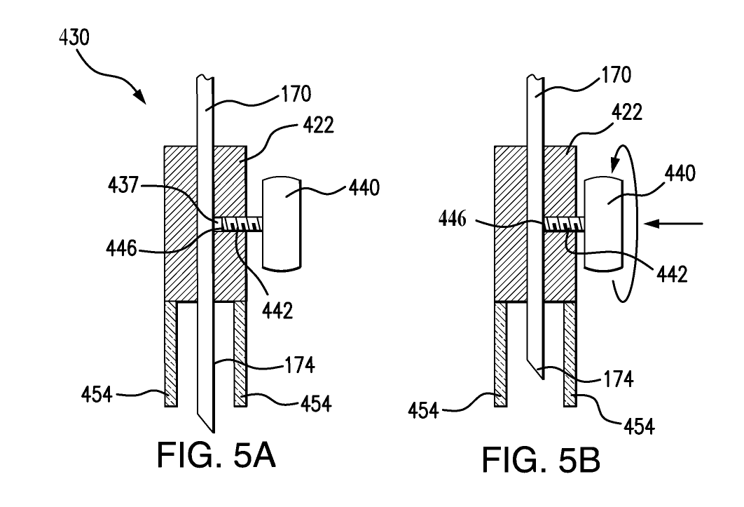

Referring to FIGS. 5A-5B, in one embodiment, a needle guard 430 of the present disclosure may include a knob 440 attached to a threaded rod 442 extending through an opening 437 formed within the housing 432. The housing may include a gasket to frictionally engage an outer surface of a needle. An inner surface of the opening 437 may include a corresponding grooved surface such that rotating the knob 440 in a first direction (e.g., clockwise) may draw the threaded rod 442 into/toward the housing 432, and rotating the knob in a second direction (e.g., counterclockwise) may draw the threaded rod 442 away from the housing 432. The knob 440 may be rotated in the second direction to a second position (FIG. 5A) in which the biopsy needle does not contact the threaded rod 442, and is free to move/slide through the needle guard, e.g., as the needle attachment component with the needle are withdrawn proximally to position the sharpened distal end 174 of the needle within the hollow tube 454 attached to or integrally formed with the housing 432. The knob 440 may be rotated in the first direction to a first position (FIG. 5B) in which a surface 446 of the threaded rod 442 is placed in contact with an outer surface of the biopsy needle 170 with sufficient force to maintain the sharpened distal end 174 of the biopsy needle 170 within the hollow tube 454 for safe removal of the tissue sample, as discussed above.

All of the devices and/or methods disclosed and claimed herein can be made and executed without undue experimentation in light of the present disclosure. While the devices and methods of this disclosure have been described in terms of preferred embodiments, it may be apparent to those of skill in the art that variations can be applied to the devices and/or methods and in the steps or in the sequence of steps of the method described herein without departing from the concept, spirit and scope of the disclosure. All such similar substitutes and modifications apparent to those skilled in the art are deemed to be within the spirit, scope and concept of the disclosure as defined by the appended claims.

What is claimed is:

1. A needle guard, comprising:
a housing;
first and second flexible arms extending distally from a proximal end of the housing;
a gasket disposed within an opening in the housing; and
a hollow tube attached to the housing between the first and second flexible arms.

2. The needle guard of claim 1, wherein a distal end of the first flexible arm includes a first tab, and a distal end of the second flexible arm includes a second tab.

3. The needle guard of claim 1, wherein the gasket includes an opening configured to slidably receive an outer surface of a needle.

4. The needle guard of claim 3, wherein the opening of the gasket is configured to frictionally engage the outer surface of the needle.

5. The needle guard of claim 1, wherein at least a portion of the hollow tube comprises an optically translucent material.

6. The needle guard of claim 1, further comprising a needle attachment component comprising a cylinder and a grip, wherein an inner surface of the cylinder is configured to frictionally engage an outer surface of the housing.

7. The needle guard of claim 2, wherein the first and second flexible arms are configured to move between a first position and a second position.

8. The needle guard of claim 7, wherein the first and second flexible arms are configured to extend through an opening formed in a proximal end of a medical device.

9. The needle guard of claim 8, wherein the first and second tabs contact an inner surface of the medical device when the first and second arms are in the first position.

10. The needle guard of claim 9, wherein the first and second tabs do not contact the inner surface of the medical device when the first and second arms are in the second position.

11. A needle guard, comprising:
a housing;
a first wing extending through a first opening in the housing, wherein a middle portion of the first wing is pivotally attached to the housing and the first wing is configured to move between a first position and a second position;
a second wing extending through a second opening in the housing, wherein a middle portion of the second wing is pivotally attached to the housing and the second wing is configured to move between a first position and a second position; and
a hollow tube attached to the housing.

12. The needle guard of claim 11, wherein the housing and hollow tube are configured to receive a needle therethrough.

13. The needle guard of claim 12, wherein a surface of the first and second wings does not contact an outer surface of the needle when in the first position.

14. The needle guard of claim 13, wherein the surface of the first and second wings contacts the outer surface of the needle when in the second position.

15. The needle guard of claim 12, wherein the first and second wings lock a portion of the needle within the housing when in the second position.

16. The needle guard of claim 15, wherein the portion includes a sharpened distal end of the needle.

17. A needle guard, comprising:
a housing;
a rod including a threaded surface extending through an opening in the housing, an inner surface of the opening including a grooved surface corresponding to the threaded surface; and
a hollow tube attached to a distal end of the housing.

18. The needle guard of claim 17, wherein the housing and hollow tube are configured to receive a needle therethrough.

19. The needle guard of claim 18, wherein rotating the rod in a first direction moves an end of the rod toward an interior of the housing, and rotating the rod in a second direction moves the end of the rod away from the interior of the housing.

20. The needle guard of claim 19, wherein rotating the rod in the first direction places the end of the rod in contact with an outer surface of the needle.
